(19) United States
(12) Patent Application Publication
(10) Pub. No.: US 2025/0375323 A1
(43) Pub. Date: Dec. 11, 2025

(54) Underwear with cannabidiol carrier

(71) Applicant: JUNG LUNG LIN, Taipei City (TW)

(72) Inventor: JUNG LUNG LIN, Taipei City (TW)

(21) Appl. No.: 18/969,108

(22) Filed: Dec. 4, 2024

(30) Foreign Application Priority Data
Jun. 7, 2024 (TW) 113206043

(51) Int. Cl. A61F 13/496; A61K 31/00

(52) U.S. Cl. CPC A61F 13/496; A61K 31/658

(57) ABSTRACT

An underwear with a cannabidiol carrier contains: a skin-friendly layer, an absorption layer, a waterproof layer, and a close-fitting fabric layer. The skin-friendly layer is fixed on an outer surface of the absorption layer to contact with user's skin, the absorption layer is arranged on an outer surface of the waterproof layer, and the waterproof layer is formed on an outer surface of the close-fitting fabric layer to contact with the personal product.

### TECHNICAL FIELD

The present disclosure relates to an underwear with a cannabidiol carrier to which cannabidiol additive is added to relieve menstrual pain.

### BACKGROUND

The main appeal of most personal care products is to have a beautiful appearance or to make it convenient for users to use them once. However, they can produce relaxing energy and repeated use effects on the body and mind, but less attention is paid to them.

Therefore, when people use personal care products, they cannot obtain cannabidiol carrier energy activation to quickly gain relaxation and further obtain health-promoting effects (such as muscle relaxation, relief of inflammation and soreness, speeding up falling asleep, extending sleep time, improving body systems, promoting metabolism, and resisting inflammation and anxiety).

The present invention has arisen to mitigate and/or obviate the afore-described disadvantages.

### SUMMARY

A primary aspect of the present invention is to provide an underwear with a cannabidiol carrier to which cannabidiol additive is added to relieve menstrual pain.

To obtain above-mentioned aspect, an underwear with a cannabidiol carrier provided by the present invention contains: a skin-friendly layer, an absorption layer, a waterproof layer, and a close-fitting fabric layer.

The skin-friendly layer is fixed on an outer surface of the absorption layer to contact with user's skin, the absorption layer is arranged on an outer surface of the waterproof layer, and the waterproof layer is formed on an outer surface of the close-fitting fabric layer to contact with the personal product.

Other objects and features will be in part apparent and in part pointed out hereinafter.

### BRIEF DESCRIPTION OF THE DRAWINGS

FIG. 1 is a plan view showing the application of an underwear with a cannabidiol carrier according to the present invention.

FIG. 2 is a perspective view showing the exploded components of the underwear with the cannabidiol carrier according to the present invention.

FIG. 3 is a cross sectional view showing the assembly of the underwear with the cannabidiol carrier according to the present invention.

FIG. 4 is another cross sectional view showing the assembly of the underwear with the cannabidiol carrier according to the present invention.

FIG. 5 is a schematic view showing the production of the waste fishing-net drawn yarns of the underwear with the cannabidiol carrier according to the present invention.

### DETAILED DESCRIPTION

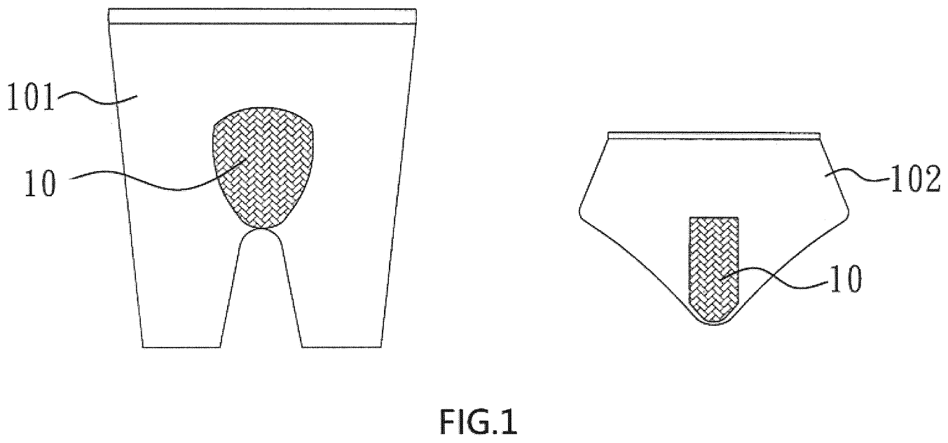

With reference to FIG. 1, an underwear with a cannabidiol carrier 10 according to a first embodiment of the present invention is adhered on a male/female menstrual pant 101 or a female menstrual pant 102.

The male/female menstrual pant 101 or the female menstrual pant 102 includes the cannabidiol carrier 10 with at least one soothing substance configured to comfort user's body and mind, wherein the cannabidiol carrier 10 is made of at least one of cannabidiol tablets, granules, powders, microcapsules containing cannabidiol oil, and antibacterial/deodorant.

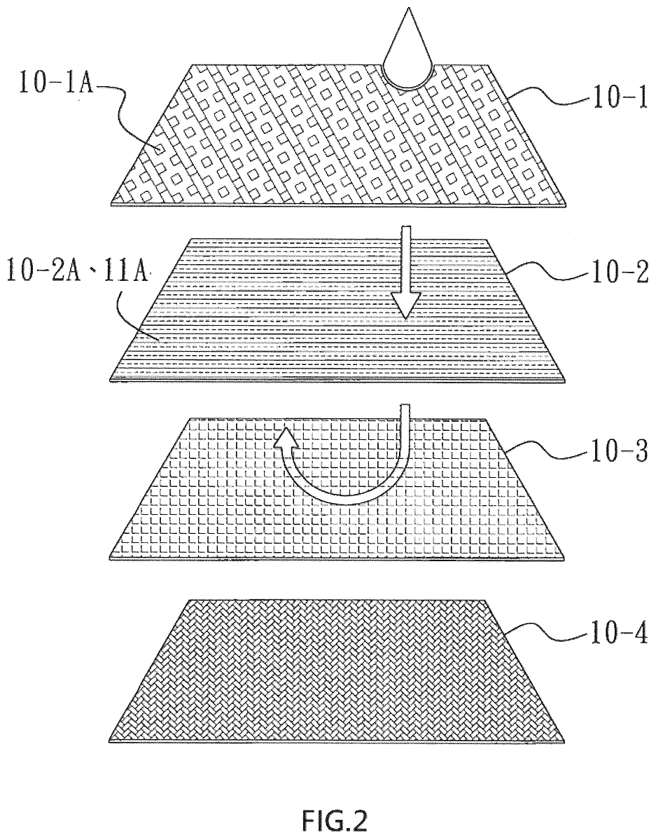

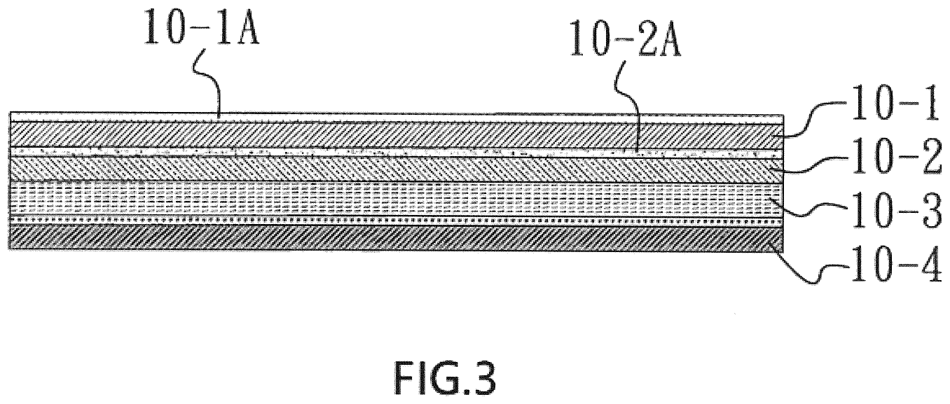

Referring to FIGS. 2 and 3, the cannabidiol carrier 10 includes multiple layers which are a skin-friendly layer 10-1, an absorption layer 10-2, a waterproof layer 10-3, and a close-fitting fabric layer 10-4, wherein the skin-friendly layer 10-1 is fixed on an outer surface of the absorption layer 10-2 to contact with the user's skin, the absorption layer 10-2 is arranged on an outer surface of the waterproof layer 10-3, and the waterproof layer 10-3 is formed on an outer surface of the close-fitting fabric layer 10-4 to contact with the personal product.

In the first embodiment, as shown in FIGS. 1-3, the skin-friendly layer 10-1 is comprised of polypropylene yarn and one-way moisture-conducting polyester fiber coated with natural antibacterial deodorant, and cannabidiol additive 10-1A is added to the skin-friendly layer 10-1, thus quickly absorbing and discharging bloods and urine from user's body to keep dry and relieve menstrual pain. Preferably, an antibacterial deodorant is added to the skin-friendly layer 10-1 to prevent bacterial growth and eliminate odors caused by urine.

The absorption layer 10-2 is accommodated in the skin-friendly layer 10-1 and is comprised of absorbent knitted fabric on which natural antibacterial agent 10-2A is coated to enhance absorptive capacity and avoid a leakage of blood and urine. The waterproof layer 10-3 is received in the absorption layer 10-2 and is comprised of a fabric mesh, polyurethane, and a breathable waterproof film to prevent the blood and the urine flowing out of the male/female menstrual pant 101 and the female menstrual pant 102. The close-fitting fabric layer 10-4 is accommodated in the waterproof layer 10-3 and contacts with the male/female menstrual pant 101 and the female menstrual pant 102, wherein the close-fitting fabric layer 10-4 is made of nylon single-sided elastic fabric to obtain using comfort. Preferably, waste fishing-net drawn yarns 15 are added to the absorption layer 10-2, the waterproof layer 10-3, and the close-fitting fabric layer 10-4.

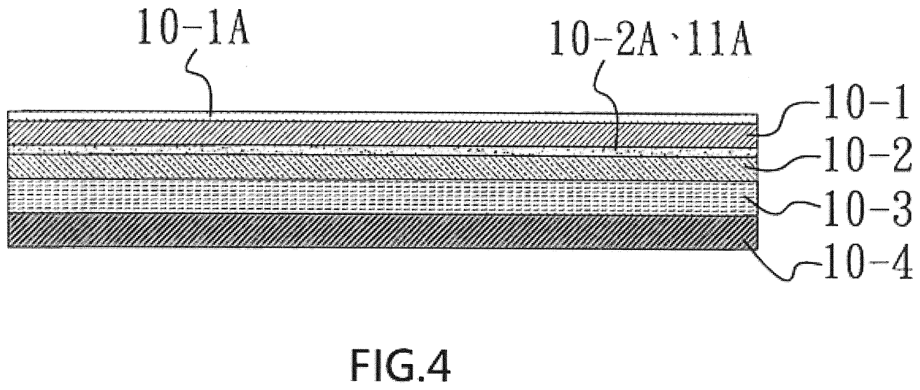

In a second embodiment, as shown in FIGS. 1, 2 and 4, the skin-friendly layer 10-1 is comprised of environmentally friendly recycled yarns and polypropylene original drawn yarns so as to contact with the user's skin. The environmentally friendly recycled yarns and the polypropylene original drawn yarns are fabrics to obtain energy-saving and environment-protecting purpose. Preferably, the polypropylene original drawn yarns have one-way moisture conduction capacity to conduct moisture downward quickly. Cannabidiol aid 11A is added to the cannabidiol carrier 10, thus soothing menstrual pains. The absorption layer 10-2 is comprised of polyester and knitted fabrics, thus accommodating bloods in the cannabidiol carrier 10 and discharging water vapors from the cannabidiol carrier 10. Preferably, natural antibacterial agents (such as biological antibacterial agents and deodorants) are used for antisepsis and deodorization. The waterproof layer 10-3 is comprised of a fabric mesh, polyurethane, and a breathable waterproof film to prevent the blood and the urine flowing out of the male/female menstrual pant 101 and the female menstrual pant 102. The close-fitting fabric layer 10-4 is made of nylon single-sided elastic fabric to obtain using comfort.

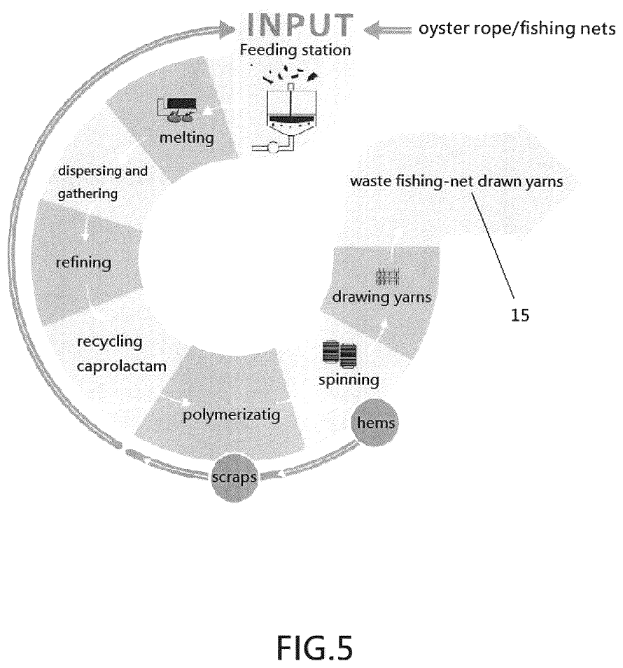

Thereby, as shown in FIG. 5, the cannabidiol carrier 10 of the present invention has advantages as follows:
A) Obtaining breathable and antibacterial purposes and reducing gynecological infections by using the skin-friendly layer 10-1, wherein the natural antibacterial agents (such as biological antibacterial agents and deodorants) are used for antisepsis and deodorization, thus eliminating odors from microorganisms and odor sources, thus significantly reducing the gynecological infections.
B) Reducing anxiety during menstrual period, wherein the cannabidiol carrier 10 completely covers groin to avoid the leakage of blood.
C) Achieving environmental friendly, wherein the waste fishing-net drawn yarns 15 are added to the absorption layer 10-2 to reduce wastes and energy consumption when dyeing.
D) The cannabidiol carrier 10 is used repeatedly to decrease a purchase cost of the personal product.
E) The cannabidiol additive 10-1A is added to the skin-friendly layer 10-1 to relieve the menstrual pain.

When introducing elements of the present invention or the preferred embodiments thereof, the articles “a”, “an”, “the” and “said” are intended to mean that there are one or more of the elements. The terms “comprising”, “including” and “having” are intended to be inclusive and mean that there may be additional elements other than the listed elements.

In view of the above, it will be seen that the several objects of the invention are achieved and other advantageous results attained.

As various changes could be made in the above constructions, products, and methods without departing from the scope of the invention, it is intended that all matter contained in the above description and shown in the accompanying drawings shall be interpreted as illustrative and not in a limiting sense.

What is claimed is:

1. An underwear with a cannabidiol carrier comprising: a skin-friendly layer, an absorption layer, a waterproof layer, and a close-fitting fabric layer;
wherein the skin-friendly layer is fixed on an outer surface of the absorption layer to contact with user's skin, the absorption layer is arranged on an outer surface of the waterproof layer, and the waterproof layer is formed on an outer surface of the close-fitting fabric layer to contact with the personal product.

2. The underwear with the cannabidiol carrier as claimed in claim 1, wherein the skin-friendly layer is comprised of polypropylene yarn and one-way moisture-conducting polyester fiber coated with natural antibacterial deodorant, and cannabidiol additive is added to the skin-friendly layer, thus quickly absorbing and discharging bloods and urine from user's body to keep dry and relieve menstrual pain;
wherein an antibacterial deodorant is added to the skin-friendly layer to prevent bacterial growth and eliminate odors caused by urine;
wherein the absorption layer is accommodated in the skin-friendly layer and is comprised of absorbent knitted fabric on which natural antibacterial agent is coated to enhance absorptive capacity and avoid a leakage of blood and urine;
wherein the waterproof layer is received in the absorption layer and is comprised of a fabric mesh, polyurethane, and a breathable waterproof film to prevent the blood and the urine flowing out of the male/female menstrual pant and the female menstrual pant;
wherein the close-fitting fabric layer is accommodated in the waterproof layer and contacts with the male/female menstrual pant and the female menstrual pant, wherein the close-fitting fabric layer is made of nylon single-sided elastic fabric to obtain using comfort.

3. The underwear with the cannabidiol carrier as claimed in claim 1, wherein the skin-friendly layer is comprised of environmentally friendly recycled yarns and polypropylene original drawn yarns so as to contact with the user's skin; the environmentally friendly recycled yarns and the polypropylene original drawn yarns are fabrics to obtain energy-saving and environment-protecting purpose;
wherein the polypropylene original drawn yarns have one-way moisture conduction capacity to conduct moisture downward quickly, natural antibacterial agents are used for antisepsis and deodorization, and cannabidiol aid is added to the cannabidiol carrier, thus soothing menstrual pains;
wherein the absorption layer is comprised of polyester and knitted fabrics, thus accommodating bloods in the cannabidiol carrier and discharging water vapors from the cannabidiol carrier;
wherein natural antibacterial agents are used for antisepsis and deodorization, the waterproof layer is comprised of a fabric mesh, polyurethane, and a breathable waterproof film to prevent the blood and the urine flowing out of the male/female menstrual pant and the female menstrual pant; and
wherein the close-fitting fabric layer is made of nylon single-sided elastic fabric to obtain using comfort.

4. The underwear with the cannabidiol carrier as claimed in claim 1, wherein the cannabidiol carrier is adhered on a male/female menstrual pant.

5. The underwear with the cannabidiol carrier as claimed in claim 1, wherein the cannabidiol carrier is adhered on a female menstrual pant.

6. The underwear with the cannabidiol carrier as claimed in claim 1, wherein waste fishing-net drawn yarns are added to the absorption layer, the waterproof layer, and the close-fitting fabric layer.
